(19) United States
(12) Patent Application Publication
(10) Pub. No.: US 2023/0027861 A1
(43) Pub. Date: Jan. 26, 2023

(54) PHOTOBIOMODULATION OF THE GASTROINTESTINAL TRACT

(71) Applicant: Mayo Foundation for Medical Education and Research, Rochester, MN (US)

(72) Inventor: Barham K. Abu Dayyeh, Rochester, MN (US)

(21) Appl. No.: 17/868,489

(22) Filed: Jul. 19, 2022

Related U.S. Application Data
(60) Provisional application No. 63/223,734, filed on Jul. 20, 2021.

(51) Int. Cl. A61N 5/06; A61B 1/00; A61B 1/06

(52) U.S. Cl. CPC A61N 5/0603; A61B 1/00082; A61B 1/0684; A61N 2005/0626

(57) ABSTRACT

Devices, systems, and methods can be used to deliver photobiomodulation therapy (PBMT) to the GI tract. For example, this document describes battery-enabled LED light source devices that can be deployed through or over an endoscope and anchored directly to the GI tract. Significant systemic effects can be produced by PBMT with application to one part of the body promoting beneficial anti-inflammatory and metabolic benefits in other remote sites promoted by enhanced mitochondrial function and mitokine signaling.

### CROSS-REFERENCE TO RELATED APPLICATIONS

This application claims the benefit of U.S. Provisional Application Ser. No. 63/223,734, filed Jul. 20, 2021. The disclosure of the prior application is considered part of (and is incorporated by reference in) the disclosure of this application.

### BACKGROUND

### 1. Technical Field

This document relates to devices and methods for delivering photobiomodulation therapy (PBMT) to the gastrointestinal (GI) tract. For example, this document relates to battery-enabled LED light source devices that can be deployed through an endoscope and anchored directly to the GI tract.

### 2. Background Information

The gut microbiome contains more than 1000 species of bacteria and viruses contributing more than 150 times the genetic material of our own genome. This diverse ecosystem is in symbiotic physiologic equilibrium with the human gut (or GI) mucosal surfaces to mediate important physiologic processes controlling food intake, nutrient absorption, energy storage and expenditure, inflammation, and critical metabolic processes through a robust immune, endocrine, enteric nervous system/vagal interaction with key organs such as the brainstem, brain, adipose tissue, liver, pancreas, and muscle. The microbiome plays an important role in human health though multiple metabolites that have important physiological functions such as short chain fatty acids, tryptophan metabolism, activation of plant polyphenols, and the production of neurotransmitters, hormones, and peptides resulting in gut mucosal integrity with robust downstream metabolomic effects.

In metabolic disease, obesity, and chronic inflammation, a state of dysbiosis results in the decrease diversity of the gut microbiome with metabolite alteration that weaken the gut epithelium integrity, increase influx of harmful bacterial metabolites resulting in chronic inflammatory states within the gut mucosa, visceral adipose tissue, liver, pancreas, cardiovascular system, and central nervous systems, which is associate with multiple human diseases such as obesity, diabetes, non-alcoholic steatohepatitis, peripheral vascular disease, coronary artery disease, inflammatory bowel disease, Parkinson's disease, Alzheimer, cancer, and multiple other diseases, where a chronic inflammatory state is contributory.

### SUMMARY

This document describes devices and methods for delivering PBMT to the GI tract. For example, this document describes battery-enabled LED light source devices that can be deployed through an endoscope and anchored directly to the GI tract.

A common sequela of metabolic disease, chronic high fat diet exposure, and resultant gut dysbiosis and chronic inflammatory state is mitochondrial fission and dysfunction within cells of the gastrointestinal tract (including intestinal stem cells) and adipose tissue resulting in production of reactive oxygen species, decrease ATP production, increase leptin resistance, decreased adiponectin that increase cardiac and CNS oxidative stress, liver gluconeogenesis, and pancreas beta-cell hyperplasia through a gut, adipose tissue, vascular system axis that transfers dysfunctional mitochondrial signals from within the gut and adipose tissue to the heart, CNS, liver, and pancreas though extracellular vesicles. PBMT or low-intensity light therapy uses light in the red or near infrared range (NIR) with wavelengths between 600 nm to 1000 nm and with a generated power ranging between 10 mW and 500 mW, for treatment of multiple inflammatory diseases. Light in this spectrum activates cytochrome c oxidase (CCO), which is unit IV of the mitochondrial respiratory chain. When CCO absorbs light, the enzyme activity is increased leading to increased electron transport, more oxygen consumption, higher mitochondrial membrane potential, and increased ATP production, thus collectively restoring damaged mitochondrial health and reversing altered mitochondrial signaling. Furthermore, light actives nonvisual phototransduction cascades involving opsins. Blue light (415 nm) and green light (540 nm) are absorbed by opsins resulting in transient receptor potential (TRP) calcium ion channels activation. Melanopsin (OPN4) is present in adipocytes, and activation via light results in downstream phototransduction cascades, which corrects protein conformation in the cell membrane and cytoskeleton, thus reversing some of the sequela of chronic inflammation and oxidative stress that could be transferred from adipose tissue to other critical organs such as the heart and brain. Indeed, electrical stimulation of white adipose tissue has shown metabolic benefits.

In one aspect, this disclosure is directed to a photobiomodulation therapy (PBMT) system. Such a PBMT system can include a PBMT device and an endoscope configured to deploy the PBMT device within a GI tract of the subject. The PBMT device can include: (i) an elongate housing including a first end portion and a second end portion opposite of the first end portion, the housing containing a battery; (ii) an LED coupled to the first end portion of the housing, the LED being powered to illuminate by the battery; and (iii) a clip mechanism coupled to the second end portion of the housing. The clip mechanism can include two opposing arms configured to pivot from an open configuration to a closed configuration. Free ends of the two opposing arms are configured to pinch tissue therebetween while in the closed configuration to secure the PBMT device to a subject.

In another aspect, this disclosure is directed to another PBMT system. The PBMT system can include a PBMT device and a clip device including two opposing arms configured to pivot from an open configuration to a closed configuration. The PBMT device includes a frame, two or more LEDs coupled to the frame, and a housing containing a battery and electronic circuitry for illuminating the two or more LEDs. Free ends of the two opposing arms of the clip device are configured to pinch tissue therebetween while in the closed configuration to secure the PBMT device to a subject.

Such a PBMT system may optionally include one or more of the following features. Each LED of the two or more LEDs may deliver red or near infrared light. A first LED of the two or more LEDs may deliver red or near infrared light, and a second LED of the two or more LEDs may deliver green or blue light.

This disclosure is also directed to a method of delivering PBMT to a subject. The method includes: a) using an endoscope to deliver and one or more of the PBMT devices described herein into the GI tract of the subject; b) anchoring the one or more of the PBMT devices to a mucosal wall of the GI tract; and c) delivering light energy from the one or more of the PBMT devices to the GI tract.

Particular embodiments of the subject matter described in this document can be implemented to realize one or more of the following advantages. The herein described endoscopic tools and accessories to allow PBMT directly to the GI tract as an adjunct treatment to rebalance the microbiome, repair damaged mitochondria within the GI tract and adipose tissues is a very appealing non-invasive method to treat or improve multiple human diseases such as obesity, diabetes, inflammatory bowel disease, cancer, Parkinson's disease, Alzheimer's, multiple sclerosis, amyotrophic lateral sclerosis, attention-deficit/hyperactivity disorder, autism and others. Such minimally invasive techniques can reduce recovery times, patient discomfort, and treatment costs.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a perspective view of a first example endoscopically deliverable battery-enabled LED PBMT device in accordance with some embodiments provided herein.

FIG. 2 is a perspective view of a second example endoscopically deliverable battery-enabled LED PBMT system in accordance with some embodiments provided herein.

FIG. 3 is a perspective view of a third example endoscopically deliverable battery-enabled LED PBMT system in accordance with some embodiments provided herein.

FIG. 4 is a perspective view of a fourth example PBMT system in accordance with some embodiments provided herein.

FIG. 5 is a perspective view of a fifth example PBMT system in accordance with some embodiments provided herein.

FIGS. 6-8 show various stages and configurations of a sixth example PBMT system in accordance with some embodiments provided herein.

FIG. 9 shows a seventh example PBMT system in accordance with some embodiments provided herein.

Like reference numbers represent corresponding parts throughout.

### DETAILED DESCRIPTION

This document describes devices and methods for delivering PBMT to the GI tract. For example, this document describes battery-enabled LED light source devices that can be deployed through an endoscope or echoendoscope and anchored directly to the GI tract or adipose tissue.

Significant systemic effects can be produced by PBMT with application to one part of the body promoting beneficial anti-inflammatory and metabolic benefits in other remote sites promoted by enhanced mitochondrial function and signaling (referred to as mitokine signaling). Although some success in gut microbiome modulation by PBMT for treatment of human disease has been demonstrated in mice using a percutaneous light sources, no available technology exists to apply PBMT directly to the gut surface and its microbiome, or to adipose tissue depots, where the effect will be enhanced given increase dose response afforded by decreased light dispersion seen with percutaneous approaches and sophisticated modulation of the gut-adipose tissue axis. In the document, multiple endoscopic tools and methods for PBMT directly in the GI tract and/or visceral adipose tissue are disclosed.

Endoscopic tools and accessories described herein allow PBMT directly to the GI tract as an adjunct treatment to rebalance the microbiome, repair damaged mitochondria within the GI tract and adipose tissues is a very appealing non-invasive method to treat or improve multiple human diseases such as obesity, diabetes, inflammatory bowel disease, cancer, Parkinson's disease, Alzheimer's, multiple sclerosis, amyotrophic lateral sclerosis, attention-deficit/hyperactivity disorder, autism and others.

This disclosure describes multiple embodiments of devices, systems, and methods that can be used to deliver PBMT to the GI tract and/or adipose tissue. For example, this disclosure describes: (i) Battery-enabled LED endoscopic clip systems that are deployed through or over an endoscope and clipped directly to the GI tract using various techniques; (ii) Trans-gastric LED light probes with an external light source modulator and an external or implantable battery for delivering PBMT through a trans-gastric approach; (iii) Trans-nasal or trans-rectal LED light probe placement with external light modulator and battery source; and (iv) LED endoscopic ultrasound with a detachable LED needle probe to deliver PBMT to the retroperitoneal, gastrohepatic ligament, mesenteric abdominal fat, or other fat depots.

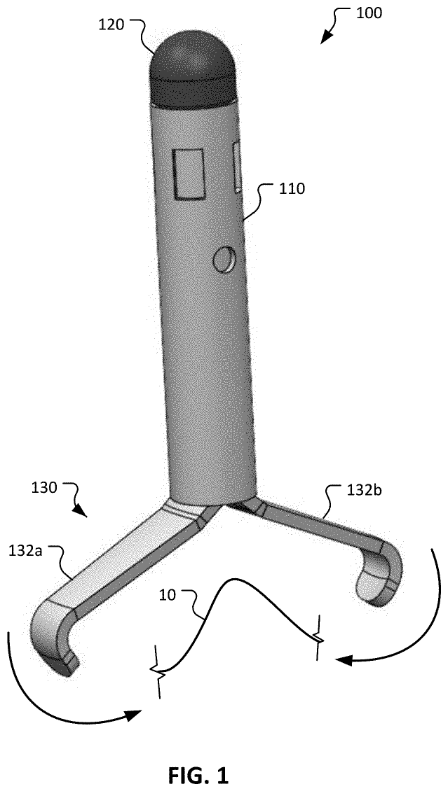

FIG. 1 illustrates an example endoscopically deliverable battery-enabled LED PBMT device 100. The PBMT device 100 can be deployed through an endoscope and clipped or anchored directly to the inner wall of the GI tract using various techniques. One technique is deploying it through the working channel of the endoscope. This can also be referred to as a “through-the-scope LED clip system.” The PBMT device 100 can be used for treatment of multiple types of inflammatory diseases within the GI tract or elsewhere, also it can illuminate the GI tract for therapeutic interventions, for example.

The PBMT device 100 includes an elongate housing 110, an LED 120, and a clip mechanism 130. The housing 110 has a first end portion and a second end portion opposite of the first end portion. In the depicted embodiment, the housing 110 is cylindrical. A battery and electronics for powering and controlling the illumination of the LED 120 are sealed within the housing 110. In some embodiments, the battery is wirelessly rechargeable via inductive charging.

The PBMT device 100 also includes the LED 120. The LED 120 is coupled to (attached to) the first end portion of the housing 110. The illumination of the LED 120 is powered by the battery and controlled by the electronics in the housing 110. In some embodiments, the LED 120 emits light in the red or near infrared (NIR) range, e.g., with wavelengths between 600 nm to 1000 nm and with a generated power ranging between 10 mW and 500 mW. Alternatively, in some embodiments the LED 120 emits blue light (with a wavelength of about 380 to 500 nm), or green light (with a wavelength of about 495 to 570 nm). In some embodiments, two or more LEDs 120 can be included on the PBMT device 100. For example, in some such embodiments one of the two or more LEDs 120 can be a red or near infrared LED, and the other one of the two or more LEDs 120 can be a green LED or a blue LED.

The PBMT device 100 also includes the clip mechanism 130. The clip mechanism 130 is coupled to (attached to) the second end portion of the housing 110. The clip mechanism 130 includes two opposing arms 132a and 132b that are configured to pivot from an open configuration (as shown) to a closed configuration. The free ends of the two opposing arms 132a-b are configured to pinch tissue 10 therebetween while secured in the closed configuration to attach the PBMT device 100 to a subject (e.g., to the mucosal wall of the subject's GI tract).

In this embodiment, the LED light source 120 is incorporated into an endoscopic clip device 100 that is deployed through or over an endoscope and is deployed to anchor on the mucosa or submucosa of the GI tract. Each PBMT device 100 can emit a particular light wavelength or a range of wavelengths. One or multiple PBMT devices 100 can be deployed to cover the desired length in the GI tract and desired light delivery spectrum. The clip/light LED system can be programmable as to emit a particulate wavelength of light during a certain time period (e.g., one wavelength during the day and another during the night)

In some embodiments, the PBMT device 100 is deployed through a working channel of an endoscope. In particular embodiments, the PBMT device 100 is deployed over the shaft of an endoscope. In this embodiment, the LED light source 120 is incorporated into an over the scope clip system that is deployed to the mucosal or sub-mucosal GI surface using suction or traction device. Once deployed the anchoring component of the clip system (e.g., the clip mechanism 130) will incorporate in the mucosa/submucosa and the LED light source 120 will emit single or multiple light wavelengths in the desired therapeutic range. In another embodiment of this system, the multiple light probes emitting a single or multiple light wave lengths are attached to the over the scope clip and supplied by a battery within the clip shaft or via nano-electrodes incorporated in the clip shaft.

Some example embodiments of the PBMT device 100 (and other PBMT devices/systems described herein) can be effectively operated in accordance with the following parameters and techniques. These example parameters and techniques are non-limiting.

1) Wavelength near infrared 660-980 nm for chromophore activation alternating with blue (415 nm), green (540 nm) lights, which impacts stem cell differentiation and activation of light-gated calcium channels rather chromophore activation; thus, achieving synergistic effects.

2) Energy density range between 1 J/cm² to 80 J/cm².

3) Mode/Frequency alternate mode between continuous and pulsed delivery. Pulsed delivery is superior except for nerve regeneration. Pulsed delivery can range between 2 Hz to 3000 Hz.

4) Low energy vs. non-coherent LED light can be used to construct the system or a combination of both.

5) Duration of treatment application: experimentally, mice treated three times per week for two weeks with NIR light (808 nm) had a 10,000-fold increase in the proportion of the beneficial bacterium (e.g., Allobaculum) in stool after 14 days of treatment with NIR light compared to sham. Thus, in some embodiments the duration of application ranges between 24 hours to 12 months with each treatment.

While the PBMT device 100 also includes the clip mechanism 130 with its two opposing arms that can closed to pinch tissue therebetween to anchor the PBMT device 100 to tissue, in some embodiments other types of anchoring mechanisms can be used for the PBMT device 100. For example, in some embodiments a helical tissue anchor can be included as part of the PBMT device 100 instead of the two opposing arms.

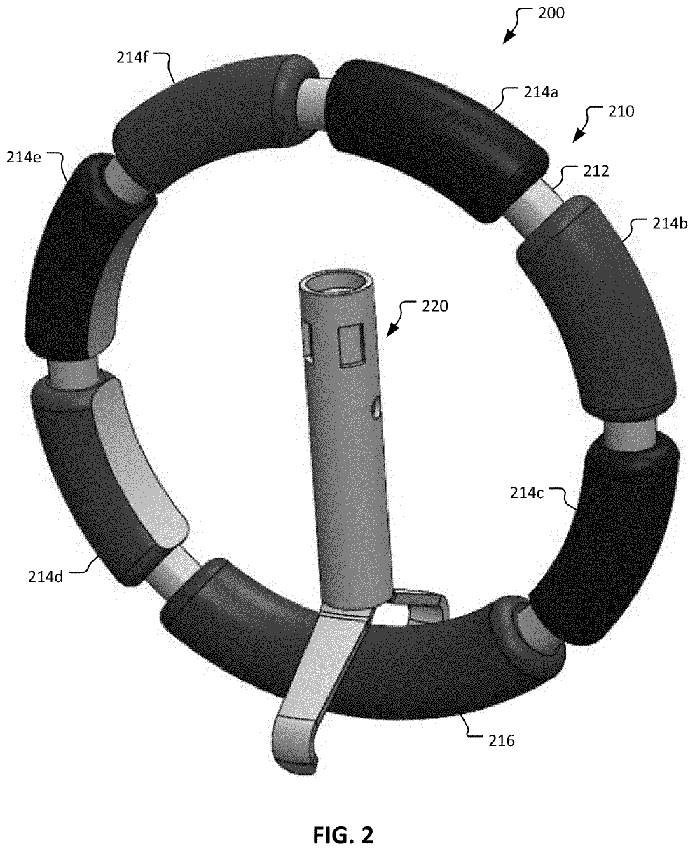

FIG. 2 illustrates another example endoscopically deliverable battery-enabled LED PBMT system 200. The LED PBMT system 200 includes an LED PBMT device 210 and a clip device 220. The clip device 220, with its two opposing clip arms that can be used to pinch tissue therebetween, can be used to attach the LED PBMT device 210 to tissue of a subject (e.g., to the mucosal wall of the subject's GI tract).

The LED PBMT device 210 includes a frame 212, multiple LED segments 214a-f, and a housing 216. In some embodiments, each of the multiple LED segments 214a-f can emit the same color/wavelength of light. In some embodiments, the multiple LED segments 214a-f can emit differing colors of light (e.g., one or more segments can emit red light, one or more segments can emit NIR light, one or more segments can emit blue light, one or more segments can emit green light, and any combination thereof).

The LED PBMT device 210 includes the housing 216 that contains an on board battery and control circuitry. In some embodiments, the battery can be wirelessly charged via inductive charging.

The LED PBMT device 210 includes the frame 212 that couples the multiple LED segments 214a-f to the housing 216. The multiple LED segments 214a-f are disposed at respective locations on the frame 212. In the depicted embodiment, the frame 212 is circular. Alternatively, in some embodiments the frame 212 can be shaped differently (e.g., U-shaped, C-shaped, rectangular, triangular, linearly, etc., without limitation).

The LED PBMT system 200 can allow manipulation of the delivered light waveforms during different times a day (e.g., night vs. day) by delivering/implanting multiple single LED PBMT devices 210 each capable of delivering a different light waveform at different programmable times during a 24 hour period, or by having a ring of sequentially-attached segmented LED lights of differing waveforms that activate based on a pre-specified program.

This LED PBMT system 200 will allow for flexibility in the energy density delivered with a titratable range (e.g., higher number of delivered clips result in higher energy density), and allow for programmable range of pulsed or continue light application by activating one of multiple LED components of the system differently (e.g., some LEDs are continuously activated throughout the duration of the treatment, and other LEDs can be pulsed at different frequencies). In addition, the duration of the treatment can be also titrated based on the battery life of the LED PBMT system 200 and the depth of the anchoring clip 220 system. Finally, the intended delivery site of the LED PBMT system 200 (and other PCMT systems described herein) can vary to include one or more of the oral cavity, esophagus, stomach, small intestine, colon, rectum, pancreas, liver, bile ducts, and/or adipose tissue, to provide a few examples.

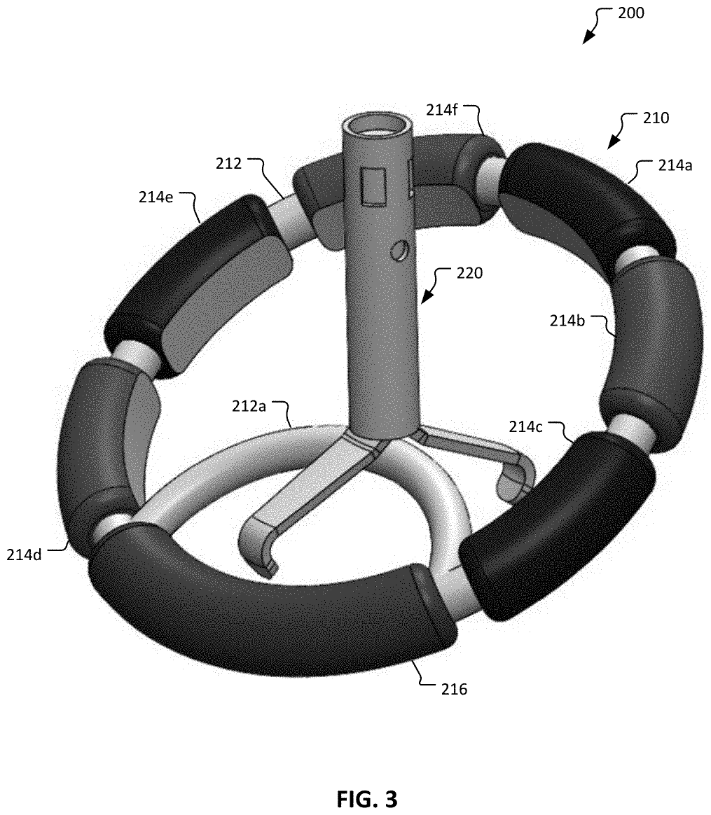

As illustrated in FIG. 3, in some embodiments the frame 212 of the LED PBMT device 210 includes a clip engagement portion 212a that is especially suited to engage with the clip device 220 for anchoring the LED PBMT device 210 to tissue and/or as a replaceable battery source. In the depicted embodiment, the clip engagement portion 212a is a semi-circular portion of the frame 212 that is within the outer periphery of the frame 212.

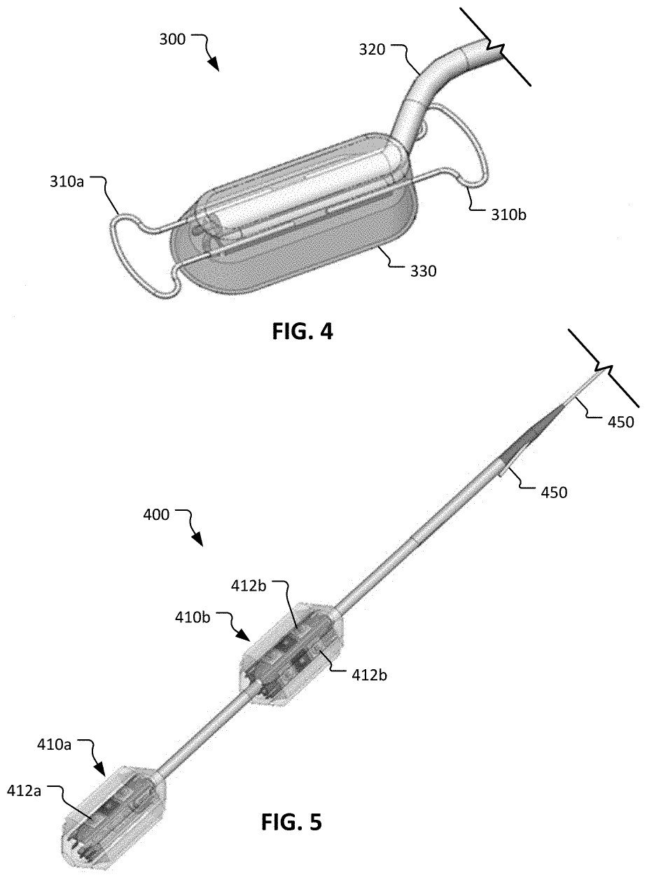

FIG. 4 illustrates another example embodiment of a PBMT system, namely PBMT system 300. The PBMT system 300 is a localized PBMT device for local gastrointestinal ulcers treatment. As shown in FIG. 4, the LED or low energy laser is in a housing 330 that includes a reflective surface that will maximize irradiance and light delivery to the ulcerated area. The PBMT system 300 has two tissue anchors 310a-b that allow clipping and elevation of the PBMT system 300 above the targeted ulcer bed. The PBMT system 300 can be powered externally through a percutaneous or subcutaneous power source, or internally through a local power source. Hence, the power cord 320 is optional.

FIG. 5 illustrates another example embodiment of a PBMT system, namely PBMT system 400. In this embodiment, sequential PBMT devices 410a and 410b are delivered over a sheet guided by a wire 450. In this iteration, each of the PBMT light sources 412a-b is housed with a reflective surface mold and the sequential devices are delivered 410a-b over a wire 450 to the targeted gastrointestinal are such as the stomach, small intestines or colon. The PBMT system 400 can be powered externally through a percutaneous or subcutaneous power source, or internally through a local power source.

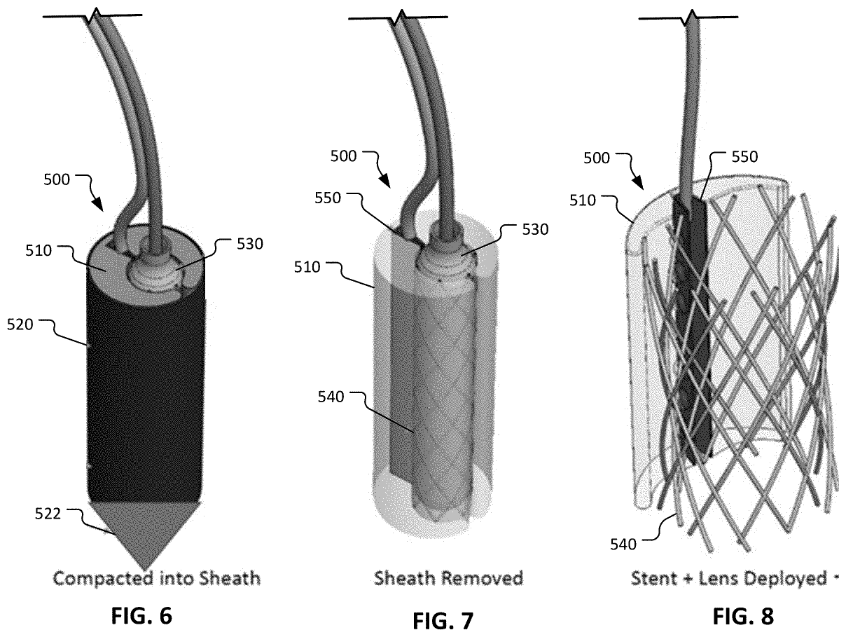

FIGS. 6-8 illustrate another example embodiment of a PBMT system, namely PBMT system 500. The PBMT system 500 includes a foldable reflector 510, a removable delivery sheath 520, an optional dilator tip 522, a balloon 530, a stent 540, and a LED light source 550. FIG. 6 shows the PBMT system 500 in a radially compact low-profile delivery configuration (within the delivery sheath 520). FIG. 7 shows the PBMT system 500 after the removal of the delivery sheath 520, but prior to radial expansion by the balloon 530. FIG. 8 shows the PBMT system 500 in an expanded operable configuration after the expansion of the balloon 530 and then the removal of the balloon 530. The expansion of the balloon 530 causes the radial enlargement of the stent 540 and the unfolding of the foldable reflector 510 (like the opening of a clamshell).

The light source 550 is coupled to the reflective surface of the foldable reflector 510. The stent 540 is also coupled to the reflective surface of the foldable reflector 510. When expanded, the stent 540 can anchor PBMT system 500 against tissue at the target GI tract segment. The light source 550 can be powered externally through a percutaneous or subcutaneous power source, or internally through a local power source.

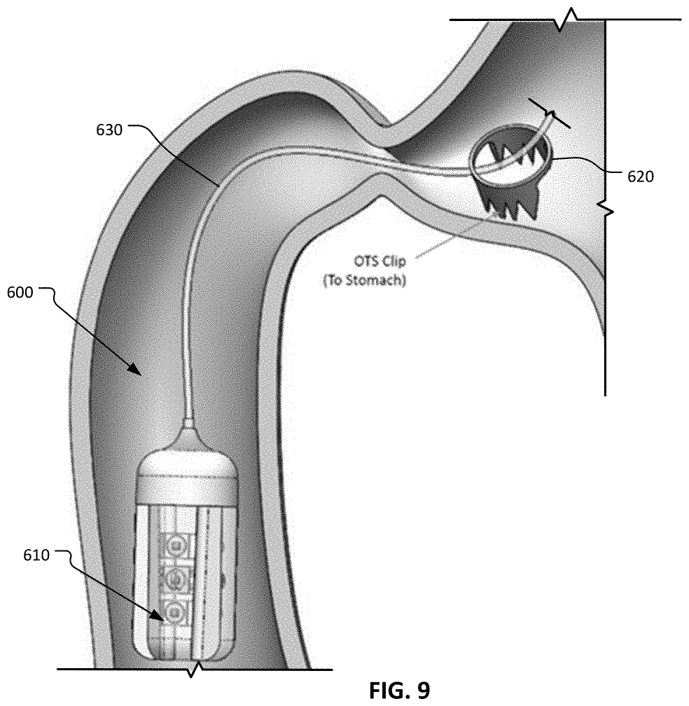

FIG. 9 illustrates another example embodiment of a PBMT system, namely PBMT system 600. In this iteration, the light source 610 is housed in a reflective capsule and anchored by a clip 620 or another anchoring mechanism in the stomach or other organs. The reflective light capsule 610 is then suspended by a flexible catheter 630 such as a spring or other material to bounce in the targeted GI tract structure and deliver light to that segment. The device can be powered externally through a percutaneous or subcutaneous power source, or internally through a local power source.

In another iteration of the device the light source is housed in a single of double pigtail configuration structure or net that is deployed in the GI tract or other abdominal structures such as omentum or visceral fat to deliver PBMT to the targeted structure or organ. The device can be powered externally through a percutaneous or subcutaneous power source, or internally through a local power source.

### ADDITIONAL EMBODIMENTS AND/OR OPTIONS

Alternatively, or additionally, PBMT can be delivered to the GI tract using systems and techniques other than the implantable PBMT systems described above. For example, in some embodiments a trans-gastric LED light probe with an external light source, modulation circuitry, and battery can be used to deliver PBMT through a trans-gastric approach. In this embodiment, a percutaneous tract will be created through standard endoscopic techniques. Once the tract has healed, single or multiple flexible LED probes can be extended to the stomach or small intestines to deliver single or multiple light wavelengths to different regions of the GI tract for PBMT. The light source can be anchored on the skin, or in the subcutaneous space. The light source can modulate the duration, pulse, and wavelength of the delivered light. The system is powered through an external battery chamber. The battery chamber can be charged wirelessly in some iterations.

In another alternative, PBMT can be delivered trans-nasally or trans-rectally using an LED light probe placement with external light modulator and battery source. In this embodiment, a single flexible LED probe, or multiple flexible LED probes, can be extended to the stomach or small intestines to deliver a single wavelength of light, or multiple light wavelengths, to different regions of the GI tract for PBMT. These are delivered through a trans-nasal or trans-rectal approach with sedated or un-sedated endoscopy or fluoroscopy. The light source modulator is anchored on a helmet or head gear. The light source can modulate the duration, pulse, and wavelength of the delivered light. The system is powered through an external battery chamber.

In another alternative, PBMT can be delivered using an LED endoscopic with ultrasound guidance and a detachable LED needle probe to deliver the PBMT to the peritoneal/mesenteric, gastrohepatic ligament, and/or visceral/retroperitoneal abdominal fat. In this embodiment, an LED needle probe is delivered through an endoscopic ultrasound needle in one or more retroperitoneal or mesenteric fat deposits. Once delivered under ultrasound guidance, the LED probe is detached from the needle apparatus into the fat tissue. A small retrieval loop is maintained in the GI lumen for future retrieval of the light probe. The battery is hosted within the light probe. Wireless battery charging is enabled in some iterations.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A photobiomodulation therapy (PBMT) system comprising:
a PBMT device comprising:
an elongate housing including a first end portion and a second end portion opposite of the first end portion, the housing containing a battery;
one or more LEDs coupled to the first end portion of the housing, the LED being powered to illuminate by the battery; and
a clip mechanism coupled to the second end portion of the housing, the clip mechanism including two opposing arms configured to pivot from an open configuration to a closed configuration, wherein free ends of the two opposing arms are configured to pinch tissue therebetween while in the closed configuration to secure the PBMT device to a subject; and
an endoscope configured to deploy the PBMT device within a GI tract of the subject.

2. A photobiomodulation therapy (PBMT) system comprising:
a PBMT device comprising:
a frame;
two or more LEDs coupled to the frame; and
a housing containing a battery and electronic circuitry for illuminating the two or more LEDs; and
a clip device including two opposing arms configured to pivot from an open configuration to a closed configuration, wherein free ends of the two opposing arms are configured to pinch tissue therebetween while in the closed configuration to secure the PBMT device to a subject.

3. The PBMT system of claim 2, wherein each LED of the two or more LEDs delivers red or near infrared light.

4. The PBMT system of claim 2, wherein a first LED of the two or more LEDs delivers red or near infrared light, and wherein a second LED of the two or more LEDs delivers green or blue light.

5. A photobiomodulation therapy (PBMT) system comprising:
a foldable reflector;
a LED light source coupled to the foldable reflector; and
an expandable stent coupled to the foldable reflector,
wherein the PBMT is reconfigurable between a low-profile deployment configuration and an expanded operable configuration.

6. The PBMT system of claim 5, further comprising a balloon within the stent while the PBMT is in the low-profile deployment configuration.

7. The PBMT system of claim 6, further comprising a delivery sheath, wherein the foldable reflector is folded within the delivery sheath while the PBMT is in the low-profile deployment configuration.

8. The PBMT system of claim 7, wherein the foldable reflector is folded around the stent and the balloon.
